(19) United States
(12) Patent Application Publication
(10) Pub. No.: US 2022/0356649 A1
(43) Pub. Date: Nov. 10, 2022

(54) WETLAID WEB COMPRISING VISCOSE FIBRE

(71) Applicants: KELHEIM FIBRES GMBH, Kelheim (DE); STORA ENSO OYJ, Helsinki (FI)

(72) Inventors: Ingo BERNT, Regensburgh (DE); Sebastian BASEL, Neuburg/Donau (DE); Laura NOUSIAINEN, Enskede (SE); Iida OLLIKAINEN, Vantaa (FI); Robin GRANKVIST, Tullinge (SE)

(21) Appl. No.: 17/765,311

(22) PCT Filed: Sep. 29, 2020
(86) PCT No.: PCT/EP2020/077252

§ 371 Date: Mar. 30, 2022

(30) Foreign Application Priority Data
Sep. 30, 2019 (EP) 19200538.7

(51) Int. Cl. D21H 13/08; D21H 21/20; D04H 1/4382; D04H 1/26; D04H 1/28

(52) U.S. Cl. CPC D21H 13/08; D21H 21/20; D04H 1/43838; D04H 1/26; D04H 1/28

(57) ABSTRACT

Wetlaid web, selected from the group consisting of wet-laid non-woven fabrics and paper, comprising a cellulosic fibre material in the form of viscose fibre at an amount of at least 5% w/w, characterized in that the wetlaid web comprises microfibrillated cellulose at an amount of 0.5% w/w to 5% w/w, wherein the microfibrillated cellulose has a particle size distribution (x₁₀) of 5 μm to 30 μm, and a wet-strength agent.

The invention relates to a wetlaid web comprising viscose fibre.

Furthermore, the invention relates to the use of the wetlaid web according to the invention as food filtration fleece or transparent cosmetic mask.

### BACKGROUND OF THE INVENTION

Viscose is a versatile material which is used in the textile industry, among others. Viscose fibres can be obtained from regenerated cellulose by the wet spinning process.

By modifying the production parameters, the viscose fibres can be imparted different properties. As a result, they can be functionalized in various ways. The functionality of the fibres is desired to be transferred to the products produced from these fibres. This requires a sufficiently high proportion of at least 5%, but usually significantly more than this, such as 10% viscose fibres or even up to 90% viscose fibres or more in the product (Here and in the following, percentage values mean weight percentages unless otherwise indicated). However, with an increasing proportion of viscose fibres the strength of products such as wet-laid non-woven fabrics and paper decreases. Therefore, the use of viscose fibres in these fields is currently limited.

With an increasing proportion of viscose fibre, in particular low wet-strength occurs. Dry paper generally exhibits a strong loss of strength when coming into contact with water, as water splits the hydrogen bonds between the fibres. A compromise of product strength and functionality has to be made. To counteract this, wet-strength agents are usually applied. These form permanent/covalent cross-links which increase the strength of paper in the wet state. However, it was observed (WO 2018/078094A1) that wet-strength agents have a reduced effect on viscose fibres compared to pulp. It is believed that this is due on the one hand to the relatively hard surface of the regenerated viscose fibres compared to natural cellulose fibres and on the other hand due to the fact that viscose fibres do not fibrillate.

As described in WO 2018/078094, the strength of cellulosic fibres, in particular viscose fibres, can be increased by treating anionic viscose fibres with cationic polymers. However, this requires an additional process step with a corresponding increase of costs.

Another known method to increase the strength of viscose fibres is to blend said fibres with Bico (bi-component) fibres. Bico fibres usually comprise a high melting polymer (e.g. PP) and a low melting polymer (e.g. PE). In the case of core-sheath Bico fibres, the low melting polymer forms the sheath and the high melting polymer is the core of the fibre. When blending with for example viscose fibres, the Bico fibres can be activated by heat, e.g. by thermocalandering. Thereby the sheath of the Bico fibres melts and joins the non-molten core with the viscose fibres. Thus, the strength of the overall structure of the nonwoven increases. A disadvantage of this method is that the resulting product is not biodegradable, in contrast to viscose fibres as such.

In EP 2 441 869 A1, it was an object to provide a fibrous sheet which has both high water disintegratability and high wet strength. The fibrous sheet disclosed in EP 2 441 869 A1 comprises unbeaten and beaten pulp, regenerated cellulose and fibrillated purified cellulose.

EP 2 781 652 A1 discloses a wet-laid nonwoven comprising long synthetic and/or natural fibres and nanofibrillar cellulose.

US 2018/0280847 A1 describes the use of a fibrillated cellulosic fibre and a stable fibre in filter media.

GB 1 064 476 A discloses a transparent paper consisting essentially of flat regenerated cellulose fibres.

US 2019/0257023 A1 relates to a modified cellulosic fibre comprising ionic moieties and a polymeric modifying agent.

### SHORT DESCRIPTION OF THE INVENTION

It is the object of the present invention to provide a wetlaid web, in particular a wet-laid non-woven fabric or a paper comprising viscose fibres and having sufficient wet strength.

The object is solved by a wetlaid web, selected from the group consisting of wet-laid non-woven fabrics and paper, comprising a cellulosic fibre material in the form of viscose fibre at an amount of at least 5% w/w, characterized in that the wetlaid web comprises microfibrillated cellulose at an amount of 0.5% w/w to 5% w/w, wherein the microfibrillated cellulose has a particle size distribution (x₁₀) of 5 μm to 30 μm, and a wet-strength agent.

The wetlaid web of the invention is suitable for the use as food filtration fleece, preferably tea bag paper, or as transparent cosmetic mask.

### DETAILED DESCRIPTION OF THE INVENTION

The present invention provides a wetlaid web comprising viscose fibre, microfibrillated cellulose and wet-strength agent.

The wetlaid web according to the present invention is selected from the group consisting of wet-laid non-woven fabrics and paper.

The wetlaid web according to the invention is suitable for example for the use as food filtration fleece or transparent cosmetic mask.

It has surprisingly been found that the addition of microfibrillated cellulose enhances the effect of wet-strength agents on viscose fibres.

Microfibrillated cellulose in general comprises long fibrillated fibres. It is believed that the long fibrils of the microfibrillated cellulose can absorb the wet-strength agent and bridge to the viscose fibres (on which wet strength agent is applied as well), thus providing a wet-strength bond between the viscose fibres by providing a substantially increased contact area. The microfibrillated cellulose apparently serves as adhesion promoter by providing additional anchoring points for the wet-strength agent, thus increasing the effect of wet-strength agent despite a high amount of viscose fibre. This affects the bonding between pulp and pulp fibres, as well as between pulp and viscose fibres, respectively between viscose and viscose fibres. Without wishing to be bound to theory, the microfibrillated cellulose might compensate for the viscose fibre in regard of the effect of a wet-strength agent. As a consequence, the amount of viscose fibres in wet-laid non-woven fabrics and paper can be increased significantly with no decrease in wet-strength or even an increase thereof.

A further advantage of the inventive wetlaid web is its origin in 100% renewable resources and its biodegradability, in contrast to wet-laid non-woven fabrics and paper blended with Bico (bi-component) fibres.

Another advantage of the inventive wetlaid web is its good pliability compared to a wetlaid web produced by the current state of the art, i.e. the folding behaviour of the inventive wetlaid web is more similar to a paper than to a spunlaced nonwoven.

An additional advantage of the inventive wetlaid web, compared to a wetlaid web produced by the current state of the art, is that the inventive wetlaid web shows essentially no linting effect, i.e. the viscose fibres are firmly bonded with the web and are not pulled out when rubbing over the web.

The viscose fibre employed in the inventive wetlaid web may be selected from the group consisting of viscose fibres with a standard cross-section, viscose fibres with a flat cross-section and mixtures thereof. The standard cross-section of viscose fibres is known to have a serrated shape as shown in the “BISFA Terminology fo Man-Made Fibres 2017” (https://www.bisfa.org/wp-content/uploads/2018/06/2017-BISFA-Terminology-final.pdf), page 23.

In the case of viscose fibres with a standard cross-section, the fibre may have a fibre length of 0.1 mm to 16 mm, preferably 3 mm to 12 mm, especially preferable 4 mm to 8 mm, and a fibre fineness of 0.5 dtex to 6.6 dtex, preferably 0.7 dtex to 1.7 dtex.

In the case of viscose fibres with a flat cross-section (in the following referred to as “viscose flat fibre”), the fibre may have a fibre fineness of 1.6 dtex to 12 dtex, preferably 1.7 dtex to 3.3 dtex.

Furthermore, in the case of a flat fibre, the cross-section of the viscose fibre may have a width-to-thickness ratio of 6:1 to 30:1.

The viscose fibre employed in the inventive wetlaid web may also be a fibre with any other cross-section, preferably trilobal or multilobal fibres. Regarding fibre length and titre, the same values as for the viscose fibres with standard cross-section apply.

In case of viscose fibre with a flat cross-section, the cross-section of the fibre may be ribbonlike as again shown in the “BISFA Terminology fo Man-Made Fibres 2017”, page 23.

In a preferred embodiment, the inventive wetlaid web is characterized in that at least a part of the comprised viscose fibre is a solid viscose fibre with a flat cross-section having the following properties:
The ratio of width B to thickness D of the fibre is 10:1 or higher.
The fibre surface is essentially smooth.
The fibre is essentially transparent.

Regarding fibre length and fineness, again the same values as for the viscose fibres with flat cross-section as per the above apply.

The term “essentially smooth” means that the fibre, apart from its edge areas, features essentially no grooves in the longitudinal direction which have a groove thickness of more than 10%, in particular more than 5%, of the fibre thickness. “Grooves” are thereby understood to indentations typical for standard viscose fibre which are small relative to the width of the fiber and are typical for standard viscose fibres, as discussed above with regard to the cross-section of viscose fibres with a standard cross-section.

Preferably, the fibre consists of cellulose by more than 98%.

The feature “consists of cellulose by more than 98%” relates to a completely dry fibre, so-called “bone dry”. Viscose fibres have in general a moisture content of 12% at normal room atmosphere.

Flat fibres of the type defined above are described in WO 2013/079305 A1 and WO 2018/158416.

A preferred type of these fibres, being a viscose flat fibre consisting of more than 98% cellulose (“bone dry” viscose flat fibre), having a ratio of width B to thickness D of 10:1 or greater and being essentially smooth and essentially transparent, is in the following, referred to as “Leonardo fibre”. As will be set out below, the Leonardo fibre is especially useful for certain applications of the inventive wetlaid web.

In a further embodiment, essentially all of the comprised viscose fibre is a flat fibre, especially the Leonardo fibre.

In a further preferred embodiment the inventive wetlaid web is characterized in that the amount of viscose fibre is 5% w/w to 95% w/w, preferably 5% w/w to 50% w/w, more preferably 10% w/w to 30% w/w.

In this preferred embodiment, the inventive wetlaid web is especially suitable as a food filtration fleece, preferably as tea bag paper.

Viscose fibres exhibit a porosity sufficient for the use as food filtration fleece. However, the wet-strength of a wetlaid web decreases with increasing amount of viscose fibres. The inventive wetlaid web is suitable for the use as food filtration fleece, due to its porosity and wet-strength conferred by the microfibrillated cellulose in combination with a wet-strength agent.

The inventive wetlaid web is especially suitable for the use as tea bag paper.

An advantage of the inventive wetlaid web is the possibility to employ viscose fibre as a substitute of abaca fibres, which are used in food filtration fleeces due to the high porosity and wet-strength of the resulting food filtration fleeces. The substitution of abaca fibres is desirable due to its low availability, especially in Europe, and its high price.

In this preferred embodiment, the use of the Leonardo fibre as defined above is advantageous.

In a further preferred embodiment the inventive wetlaid web is characterized in that the amount of viscose fibre is 50% w/w and more, preferably 80% w/w and more, more preferably 95% w/w and more.

Thus, this embodiment is characterized by a very high amount of viscose fibre contained in the web. These embodiments are especially useful for the use of the inventive wetlaid web in a transparent cosmetic mask.

A crucial quality attribute of cosmetic masks, especially of face masks, is a high transparency in wet state. This can be especially achieved with the Leonardo fibres as described above.

A high proportion of Leonardo fibres in the cosmetic mask is required to achieve an optimum effect. However, it was found that a wet-laid non-woven fabric with a high amount of Leonardo fibres has an insufficient wet-strength. This, again, seems to be due to the low effect of wet-strength agents on viscose fibres. However, sufficient wet-strength of wet-laid non-woven fabric used as cosmetic mask are a necessity for a satisfactory handling by the user.

An advantage of the inventive wetlaid web comprising the Leonardo fibres is its suitability as cosmetic mask, due to the high transparency of Leonardo fibres and the wet-strength conferred by the microfibrillated cellulose in combination with a wet-strength agent.

In a further preferred embodiment the inventive wetlaid web is characterized in that the wetlaid web comprises in addition to the viscose fibre further cellulosic fibre material.

The further cellulosic fibre material may be selected from the group consisting of wood pulp, cotton, abaca, sisal, hemp and kenaf. Wood pulp is a preferred material.

The wood pulp may be hardwood or softwood chemical, chemi-mechanical, chemi-thermomechanical or mechanical pulp.

The wetlaid web of the invention may comprise in addition to the viscose fibre non-cellulosic fibres selected from the group consisting of polyester, polypropylene, polyethylene, aramid, glass fibre and carbon fibre.

However, in a further preferred embodiment the inventive wetlaid web is characterized in that the fibre material comprised in the wetlaid web essentially consists of cellulosic fibre material.

Thus, the wetlaid web preferably contains no fibre material of synthetic polymers.

The wetlaid web according to the invention is characterized in a preferred embodiment in that the microfibrillated cellulose has a particle size distribution (x₁₀) of 10 μm to 30 μm, preferably 12 μm to 28 μm, more preferably 12 μm to 25 μm.

Microfibrillated cellulose (MFC) comprises partly or totally of fibrillated cellulose or lignocellulose fibres. The actual fibril diameter or particle size distribution and/or aspect ratio (length/width) depends on the source and the manufacturing methods.

The smallest fibril is called elementary fibril having a diameter of approximately 2-4 nm (see e.g. Chinga-Carrasco, G.; Cellulose fibres, nanofibrils and microfibrils: The morphological sequence of MFC components from a plant physiology and fibre technology point of view, Nanoscale research letters 2011, 6:417). The aggregated form of elementary fibrils, also defined as microfibril (Fengel, D., Ultrastructural behavior of cell wall polysaccharides, Tappi J., March 1970, Vol 53, No. 3.), is the main product obtained when producing MFC, e.g. by using an extended refining process or pressure-drop disintegration process. The length of the fibrils can vary from around 1 μm to 10 μm or more.

As can be understood from the above, MFC is no “cellulosic fibre material” in the sense of the present invention.

A coarse MFC grade might contain a substantial fraction of fibrillated fibres, i.e. protruding fibrils from the tracheid (cellulose fibre), and with a certain amount of fibrils liberated from the tracheid (cellulose fibre).

MFC can also be characterized by various physical or physical-chemical properties such as its ability to form a gel-like material at low solid content (1-5 wt %) when dispersed in water, or a large surface area. The cellulose fibre is preferably fibrillated to such an extent that the final specific surface area of the formed MFC is from about 1 to about 200 m²/g, preferably 50-200 m²/g, when determined for a freeze-dried material with the BET method.

Various methods exist to produce MFC, such as single or multiple pass refining, pre-hydrolysis followed by refining or high shear disintegration or liberation of fibrils. One or several pre-treatment steps are usually required in order to make MFC manufacturing both energy efficient and sustainable. The cellulose fibres of the pulp to be supplied may thus be pre-treated enzymatically or chemically, for example to reduce the quantity of hemicellulose or lignin. The cellulose fibres may be chemically modified before fibrillation, wherein the cellulose molecules contain functional groups other (or more) than present in the original native cellulose. Such functional groups include, among others, carboxymethyl (CMC), aldehyde and/or carboxyl groups (cellulose obtained by N-oxyl mediated oxydation, for example catalyzed by TEMPO), or quaternary ammonium (cationic cellulose). After modification or oxidation according to one of the above-described methods, it is easier to disintegrate the fibres to produce MFC. Mechanical disintegration of the pre-treated fibres, e.g. hydrolysed, pre-swelled, or oxidized cellulose raw material is carried out with suitable equipment such as a refiner, grinder, homogenizer, colloider, friction grinder, ultrasound sonicator, fluidizer such as microfluidizer, macrofluidizer or fluidizer-type homogenizer.

MFC may contain hemicelluloses. The amount is dependent on the plant source. Depending on the MFC manufacturing method, the product might also contain fines, or nanocrystalline cellulose or e.g. other chemicals present in wood fibres or in papermaking process. The product might also contain various amounts of micron size fibre particles that have not been efficiently fibrillated.

MFC can be produced from wood cellulose fibres, from hardwood as well as from softwood fibres. It can also be made from microbial sources, agricultural fibres such as wheat straw pulp, bamboo, bagasse, or other non-wood fibre sources. It is preferably made from pulp including pulp from virgin fibre, e.g. mechanical, chemical and/or thermomechanical pulps. It can also be made from broke or recycled paper.

The above described definition of MFC includes, but is not limited to, the new proposed TAPPI standard W13021 on cellulose nanofibril or microfibril (CMF) defining a cellulose nanofibre material containing multiple elementary fibrils with both crystalline and amorphous regions, having a high aspect ratio with width of 5-30 nm and an aspect ratio usually greater than 50.

Four different types of MFC, in the following designated as “MFC 1”, “MFC 2”, “MFC 3” and “MFC 4” are discussed in the following in more detail. All types of MFC are non-modified microfibrillated cellulose material produced from wood pulp. MFC 2 is a finer material consisting of smaller fibrils compared to MFC 1, MFC 3 and MFC 4, as can be seen below in Table 1. MFC 3 shows the highest particle size distribution.

TABLE 1

| MFC | | | Particle size distribution | |
|---|---|---|---|---|
| Type | Concentration | pH | x₁₀ | x₉₀ |
| MFC 1 | 2.5-4.0 wt % | 6.0-8.0 | 12-25 μm | 220-720 μm |
| MFC 2 | 3.0-4.5 wt % | 6.0-8.0 | 6-14 μm | 35-115 μm |
| MFC 3 | 2.5-4.0 wt % | 6.0-8.0 | 30-35 μm | 1100-1400 μm |
| MFC 4 | 2.5-4 wt % | 6.0-8.0 | 25-28 μm | 720-1000 μm |

Particle size distribution was measured with Malvern Mastersizer 3000 based on laser diffraction. The intensity of light scattered from particles was measured and the corresponding size of the particles was calculated. This measurement technology is based on the standard ISO 13320:2009 with the following settings on the equipment used:
Blue and red laser used
Particle type: non-spherical, based on Mie Theory (for both lasers)
Refractive index: 1.5
Absorption index: 0.01
Density: 1.6 g/cm³
Refractive index of dispersant (water): 1.33
Level sensor threshold: 100
Measurement times for red and blue laser—background: 20 s—sample: 10 s. 5 measurements with delay between 5 s
Pre-measurement delay: 60 s
Obscuration limit: 0.5-8%
Stirrer speed during measurement: 2600 rpm
Ultrasound with 30% for 90 s before measurement in pulse mode with 30 s on, 30 s off
Degasing function activated after filling of the tank and ultrasonic treatment
Auto cleaning with 3 cycles with degasing, no ultra during cleaning
Result type: Volume distribution

This equipment can measure particles in the range of 0.01-3500 μm. It is noted that this equipment assumes spherical particles.

The amount of wet-strength agent contained in the wetlaid web according to the invention may be 0.1% w/w to 5% w/w, preferably 0.5% w/w to 3% w/w, especially 1% w/w to 3% w/w.

As a wet-strength agent, any wet-strength agent commonly used for in paper production can be employed.

In a preferred embodiment the inventive wetlaid web is characterized in that the wet-strength agent is selected from the group consisting of polyamine epichlorhydrin resins (PAE) and polyamidamine epichlorhydrin resins (PAAE).

The wet-strength agent may be a polymeric, preferably a cationic, chemical compound selected from the group consisting of hydrophilized polyisocyanates, glyoxalized polyacrylamides or melamine-formaldehyde resins. Preferred wet-strength agents are epichlorhydrin resins, more preferably polyamine epichlorhydrin resins (PAE) or polyamidamine epichlorhydrin resins (PAAE).

In a preferred embodiment the inventive wetlaid web is characterized in that the value of the wet-strength of the wetlaid web is at least 18%, preferably at least 20% of the value of the dry-strength.

The wet-strength and the dry-strength of the wetlaid web may be calculated by the following formula: (strength MD+strength CD)/2. MD is the abbreviation for machine direction. “Strength MD” stands for the strength in machine direction. CD is the abbreviation for cross direction. “Strength CD” stands for the strength in cross direction.

Strength MD and CD may be measured by a usual tension test.

The effect of the MFC to enhance the action of wet-strength agents on viscose fibres can also be used for making various types of functionalized wetlaid webs. Further examples besides the use as food filtration fleece and transparent cosmetic mask are the use as flame-retarding fleece, high dye absorption fleece and intrinsic water repellency fleece. Therefore, differing amounts of the viscose fibre with the intrinsic function in the inventive wetlaid web may be used. Flame-retardant fibres such as the viscose fibre Danufil BF (marketed by Kelheim Fibres GmbH) may be used in a flame-retarding fleece. Viscose fibre with high dye absorption, such as the viscose fibre Danufil Deep Dye (marketed by Kelheim Fibres GmbH) may be used in a fleece with high dye absorption properties. Water-repellent viscose fibres such as the viscose fibre Olea (marketed by Kelheim Fibres GmbH) may be used in an intrinsic water repellency fleece.

For the purposes of making a functionalized wetlaid web, the above-mentioned fibres with intrinsic functions can be mixed with non-modified viscose fibre to set the desired degree of functionalization in the resulting fleece.

It has surprisingly been found that the production of the inventive wetlaid web does not need to substantially deviate from the usual process steps of wet-laid non-woven fabrics or paper production. It does therefore not pose any special process requirements and is uncomplicated.

### EXAMPLES

The following examples further illustrate the inventive wetlaid web and its preferred embodiments.

### Example 1

An overview of the composition of the samples of example 1 is shown in Table 2.

TABLE 2
Composition of samples of example 1

| Sample | 1a | 1b | 1c | 1d |
|---|---|---|---|---|
| Pulp | Canfor ECF 90 | — | — | — |
| Amount of pulp [wt %] | 100 | — | — | — |
| Viscose fibre | — | Danufil | Danufil | Olea |
| | | 1.7 dtex/5 mm | 1.7 dtex/5 mm | 1.7 dtex/5 mm |
| Amount of viscose | — | 98 | 96 | 96 |
| fibre [wt %] | | | | |
| Type/Amount of MFC* | —/0 | “MFC 1”/2 | “MFC 1”/4 | “MFC 1”/4 |
| [wt %] | | | | |

*MFC = Microfibrillated cellulose

Production of Web Samples:

Wet-laid non-woven fabrics were produced on an inclined wire paper machine, manufactured by the company Pill Nassvliestechnik. Danufil is a standard viscose short cut fibre (with standard cross-section). Olea is a functionalized viscose fibre with standard cross-section and hydrophobic properties. Microfibrillated cellulose (MFC) from StoraEnso, i.e. MFC 1, was used.

Pulp (Canfor ECF 90) was pulped in a pulper and transferred to the pulp vat in sample 1a). Viscose fibres and microfibrillated cellulose were added directly to a container (vat) in samples 1b) to 1d). After 5 minutes stirring in the vat, the production of the web was started. The concentration of the fibre material (viscose+pulp) in the container was 1 g/L and the concentration of the fibre material (viscose+pulp) in the fibre suspension at the beginning of the web production was 0.26 g/L. The wet-laid non-woven fabrics were produced with a belt speed of 4 m/min and a basis weight target of 65 g/m².

With the same parameters, it was not possible to produce a wet-laid non-woven fabric with 100% viscose fibres suitable for further testing, as it had no strength and could not be handled accordingly for further testing.

Testing of Web Samples:

The produced wet-laid non-woven fabrics were tested with regard to the parameters strength, width of the test specimen being 1.5 cm, and thickness. Strength index was calculated with the following formula: strength index=strength [cN]/basis weight [g/m²]. The results are shown in Table 3.

TABLE 3
Testing results of samples of example 1

| Sample | 1a | 1b | 1c | 1d |
|---|---|---|---|---|
| Tensile strength index MD [cN*m²/g] | 11.9 | 5.1 | 20.4 | 18.0 |
| Tensile strength index CD [cN*m²/g] | 7.8 | 4.8 | 10.7 | 8.7 |
| Basis weight [g/m²] | 59 | 64 | 66 | 66 |
| Air permeability [L/m²s] | 406 | 1934 | 1626 | 1420 |
| Thickness [mm] | 0.395 | 0.518 | 0.557 | 0.494 |

Sample 1a) serves as reference sample for a good paper and wet-laid non-woven fabric quality which is suitable/good enough for further processing.

Sample 1b): As already mentioned, it was not possible to produce a wetlaid web product with 100% viscose fibres, which was still suitable for testing. However, already the addition of 2 wt % microfibrillated cellulose to the standard viscose short cut fibre Danufil resulted in a well manageable web with sufficient strength. It is known that viscose fibres are used to adjust porosity in non-woven fabrics. As expected, the web produced had a very high air permeability.

Sample 1c): An increase of added microfibrillated cellulose from 2 wt % to 4 wt % led to an increase in strength of the non-woven fabric which was 50% higher than that of the reference sample 1a). Thus it even exceeded the requirements. At the same time, the air permeability of the sample was excellent and even four times as high as of the reference sample 1a).

Sample 1d): The functionalized viscose fibre Olea was used. The produced web with 4 wt % microfibrillated cellulose had a very good strength and air permeability. In addition, the functionality of the fibre (hydrophobicity) was measureable in the non-woven as well. The non-woven fabric was completely hydrophobic, which was due to the high amount of Olea fibre.

The hydrophobicity of fibre and wetlaid web was tested as follows: A bundle with a diameter of approximately 2 cm was formed from fibres or web. The bundle was placed on a water surface. The product was considered hydrophobic if the bundle floated on the water surface for at least 24 hours without wetting.

### Example 2

An overview of the composition of the samples of example 2 is shown in Table 4.

TABLE 4
Composition of samples of example 2

| Sample | 2a | 2b | 2c |
|---|---|---|---|
| Viscose fibre | Leonardo | Leonardo | Leonardo |
| | 9 dtex/6 mm | 2.5 dtex/5 mm | 2.5 dtex/5 mm |
| Amount of viscose | 98 | 100 | 98 |
| fibre [wt %] | | | |
| Type/Amount of | “MFC 1”/2 | —/0 | “MFC 1”/2 |
| MFC* [wt %] | | | |
| Wet-strength agent | Yes | Yes | Yes |

*MFC = Microfibrillated cellulose

Production of Web Samples:

Leonardo fibre as defined above was employed in this example. Microfibrillated cellulose (MFC) from StoraEnso, i.e. MFC 1, was used. The wet-strength agent used was Fennostrength 505 (PAE) from Kemira (30 g/kg viscose material), viscose material being defined as 100 wt % of viscose fibre+MFC).

Viscose fibres and microfibrillated cellulose were added directly to the container (vat) in sample 2a) to 2c). Subsequently, for samples 2a) and 2c), the wet-strength agent was added in the vat. After 5 minutes stirring in the vat, the production of the fleece was started. The concentration of the fibre material (viscose) in the container was 1 g/L and the concentration of the fibre material (viscose) in the fibre suspension at the beginning of the web production was 0.17 g/L. The wet-laid non-woven fabrics were produced with a belt speed of 4 m/min and a basis weight target of 45 g/m², respectively 20 g/m².

Testing of Web Samples:

The produced wet-laid non-woven fabrics were tested with regard to the parameter strength, width of the test specimen being 1.5 cm. Strength index was calculated with the following formula: strength index=strength [cN]/basis weight [g/m²]. The results are shown in Table 5.

TABLE 5
Testing results of samples of example 2

| | | | | Δ 2b) |
|---|---|---|---|---|
| Sample | 2a | 2b | 2c | vs 2c) |
| Tensile strength index MD-dry [cN*m²/g] | 12.2 | 16.0 | 20.0 | +24 |
| Tensile strength index CD-dry [cN*m²/g] | 5.9 | 6.4 | 9.1 | +42 |
| Tensile strength index MD-wet [cN*m²/g] | — | 2.6 | 9.1 | +48 |
| Tensile strength index CD-wet [cN*m²/g] | — | 1.1 | 4.0 | +60 |
| Basis weight [g/m²l | 45 | 20 | 20 | — |

Sample 2a): The highly transparent viscose fibre Leonardo was used. Due to the high proportion of viscose fibre, the non-woven fabric from sample 2a) showed good transparency, while having a good dry-strength at the same time, as compared with reference sample 1a). The non-woven fabric also showed good haptic strength and dimensional stability in the wet state, as required for example for cosmetic face masks.

This is particularly surprising as the person skilled in the art knows that wet-strength agents in combination with viscose fibres only show very limited effectiveness. This is due on the one hand to the relatively hard surface of the regenerated viscose fibres compared to natural cellulose fibres and on the other hand due to the fact that the viscose fibres do not fibrillate. Therefore, there is little contact area between the fibres and wet-strength agents, which can thus only produce a few bonds between the fibres.

Samples 2b) and 2c): To evaluate/confirm the effect shown in sample 2a), a sample of a 20 g/m² non-woven fabric was produced one time with and one time without microfibrillated cellulose.

The test showed that even sample 2b) without microfibrillated cellulose had a relatively good strength due to the special structure of the Leonardo fibre. The addition of microfibrillated cellulose in sample 2c) further increased the strength, but surprisingly the wet-strength increased twice as much as the dry-strength of the non-woven.

This effect is attributed to the interaction of microfibrillated cellulose and wet-strength agent. The long fibrils of the microfibrillated cellulose can absorb the wet-strength agent and bridge to the viscose fibres (on which wet-strength agent is applied as well), thus providing a wet-strength bond between the viscose fibres by providing a substantially increased contact area. The microfibrillated cellulose serves as adhesion promoter by providing additional anchoring points for the wet-strength agent.

### Example 3

An overview of the composition of the samples of example 3 is shown in Table 5.

TABLE 6
Composition of samples of example 3

| Sample | 3a | 3b | 3c |
|---|---|---|---|
| Pulp | Canfor ECF 90 | Canfor ECF 90 | Canfor ECF 90 |
| Amount of pulp | 80 | 80 | 80 |
| [wt %] | | | |
| Viscose fibre | Viloft | Viloft | Viloft |
| | 1.9 dtex/10 mm | 1.9 dtex/10 mm | 1.9 dtex/10 mm |
| Amount of viscose | 20 | 19 | 19 |
| fibre [wt %] | | | |
| Type/Amount of | —/0 | “MFC 2”/1 | “MFC 1”/1 |
| MFC* [wt %] | | | |

*MFC = Microfibrillated cellulose

Production of Web Samples:

Wet-laid non-woven fabrics were produced on an inclined wire paper machine, manufactured by the company Pill Nassvliestechnik. Viloft® is a flat viscose short cut fibre available from Kelheim Fibres GmbH. Microfibrillated cellulose (MFC) from StoraEnso, i.e. MFC 1 and MFC 2 according to Table 1 was used.

Pulp (Canfor ECF 90) was pulped in a pulper and transferred to the pulp vat. Viscose fibres (samples 3a) to 3c)) and microfibrillated cellulose (samples 3b) and 3c)) were added directly to a container (vat). After 5 minutes stirring in the vat, the production of the web was started. The concentration of the fibre material (viscose+pulp) in the container was 1 g/L and the concentration of the fibre material (viscose+pulp) in the fibre suspension at the beginning of the web production was 0.39 g/L. The wet-laid non-woven fabrics were produced with a belt speed of 4 m/min and a basis weight target of 65 g/m².

Testing of Web Samples:

The produced wet-laid non-woven fabrics were tested with regard to the parameters strength, width of the test specimen being 1.5 cm, air permeability and thickness. Strength index was calculated with the following formula: strength index=strength [cN]/basis weight [g/m²]. The results are shown in Table 6.

TABLE 6
Testing results of samples of example 3

| Sample | 3a | 3b | 3c |
|---|---|---|---|
| Tensile strength index MD [cN*m²/g] | 12.2 | 15.2 | 18.0 |
| Tensile strength index CD [cN*m²/g] | 6.8 | 7.3 | 8.2 |
| Basis weight [g/m²] | 63 | 62 | 62 |
| Air permeability [L/m²s] | 406 | 412 | 375 |
| Thickness [mm] | 0.395 | 0.428 | 0.419 |

Sample 3a) was produced without addition of MFC and serves as reference sample for a good paper and wet-laid non-woven fabric quality which is suitable/good enough for further processing.

Sample 3b), 3c): Both samples show a significant increase in tensile strength compared to the reference sample 3a), produced without the addition of MFC.

The sample 3b) with addition of MFC 2 shows a smaller increase in tensile index MD (+24.6%) versus the reference sample 3a) than the sample 3c) versus the reference sample 3a). Sample 3c) was produced with addition of MFC 1 (+47.5% tensile index MD). The results demonstrate that both, MFC 1 and MFC 2, lead to an increase in tensile index MD of the reference sample.

### Example 4

An overview of the composition of the samples of example 4 is shown in Table 7.

TABLE 7
Composition of samples of example 4

| Sample | 4a | 4b |
|---|---|---|
| Pulp | Canfor ECF 90 | Canfor ECF 90 |
| Amount of pulp [wt %] | 80 | 79 |
| Viscose fibre 1 | Danufil | Danufil |
| | 0.9 dtex/5 mm | 0.9 dtex/5 mm |
| Amount of viscose fibre 1 [wt %] | 13 | 12 |
| Viscose fibre 2 | Danufil | Danufil |
| | 1.7 dtex/4 mm | 1.7 dtex/4 mm |
| Amount of viscose fibre 2 [wt %] | 7 | 6 |
| Type/Amount of MFC* [wt %] | —/0 | “MFC 1 /3 |
| Wet-strength agent | Yes | Yes |

*MFC = Microfibrillated cellulose

Production of Web Samples:

Wet-laid non-woven fabrics were produced on an inclined wire paper machine, manufactured by the company Pill Nassvliestechnik. Danufil® is a viscose short cut fibre available from Kelheim Fibres GmbH. Microfibrillated cellulose (MFC) from StoraEnso, i.e. MFC 1 according to Table 1 was used. The wet-strength agent used was Giluton 20XP (PAAE) from Kurita (10 g/kg viscose material), viscose material being defined as 100 wt % of viscose fibre+MFC.

Pulp (Canfor ECF 90) was pulped in a pulper and transferred to the pulp vat. Viscose fibres (samples 4a) and 4b)) were added directly to a container (vat). Microfibrillated cellulose was also pulped in a pulper and then added to the container (vat) (sample 4b)). After 5 minutes stirring in the vat, the production of the web was started. The concentration of the fibre material (viscose+pulp)+the MFC in the container was 1 g/L and the concentration of the fibre material (viscose+pulp)+the MFC in the fibre suspension at the beginning of the web production was 0.18 g/L. The wet-laid non-woven fabrics were produced with a belt speed of 4 m/min and a basis weight target of 30 g/m².

Testing of Web Samples:

The produced wet-laid non-woven fabrics were tested with regard to the parameters strength, width of the test specimen being 1.5 cm, air permeability and thickness. Strength index was calculated with the following formula: strength index=strength [cN]/basis weight [g/m²]. The results are shown in Table 8.

TABLE 8
Testing results of samples of example 4

| Sample | 4a | 4b |
|---|---|---|
| Tensile strength index MD-dry [cN*m²/g] | 7.4 | 20.4 |
| Tensile strength index CD-dry [cN*m²/g] | 5.1 | 14.7 |
| Tensile strength index MD-wet [cN*m²/g] | 2.8 | 6.9 |
| Basis weight [g/m²] | 31 | 28 |
| Air permeability [L/m²s] | 1352 | 978 |
| Thickness [mm\| | 0.237 | 0.198 |

Sample 4a) was produced without addition of MFC and serves as reference sample for a good paper and wet-laid non-woven fabric quality which is suitable/good enough for further processing.

Sample 4b): This sample shows a significant increase in dry tensile strength (280%) and in wet tensile strength (250%) compared to the reference sample 4a), produced without the addition of MFC.

Example 4 showed that the microfibrillated cellulose serves as adhesion promoter also in the wet state of viscose fibres with standard cross-section, by providing additional anchoring points for the wet-strength agent.

### Example 5

An overview of the composition of the samples of example 5 is shown in Table 9.

TABLE 9
Composition of samples of example 5

| Sample | 5a | 5b | 5c | 5d |
|---|---|---|---|---|
| Pulp | Canfor ECF 90 | Canfor ECF 90 | Canfor ECF 90 | Canfor ECF 90 |
| Amount of pulp [wt %] | 80 | 78 | 78 | 78 |
| Viscose fibre | Danufil | Danufil | Danufil | Danufil |
| | 1.7 dtex/5 mm | 1.7 dtex/5 mm | 1.7 dtex/5 mm | 1.7 dtex/5 mm |
| Amount of viscose | 20 | 19 | 19 | 19 |
| fibre [%] | | | | |
| Type/Amount of MFC* | —/0 | “MFC 1”/3 | “MFC 3”/3 | “MFC 4”/3 |
| [wt %] | | | | |
| Wet-strength agent | Yes | Yes | Yes | Yes |

*MFC = Microfibrillated cellulose

Production of Web Samples:

Wet-laid non-woven fabrics were produced on an inclined wire paper machine, manufactured by the company Pill Nassvliestechnik. Danufil is a standard viscose short cut fibre (with standard cross-section). Microfibrillated cellulose (MFC) from StoraEnso was used. The wet-strength agent used was Giluton 20XP (PAAE) from Kurita (10 g/kg viscose material), viscose material being defined as 100 wt % of viscose fibre+MFC).

Pulp (Canfor ECF 90) was pulped in a pulper and transferred to the pulp vat. Microfibrillated cellulose was pulped and added to the container (vat) in samples 5b) to 5d).

Viscose fibres were then added to the container (vat) in samples 5b) to 5d). After 5 minutes stirring in the vat, the production of the web was started. The concentration of the fibre material (viscose+pulp) in the container was 1 g/L and the concentration of the fibre material (viscose+pulp) in the fibre suspension at the beginning of the web production was 0.24 g/L. The wet-laid non-woven fabrics were produced with a belt speed of 4 m/min and a basis weight target of 40 g/m².

Testing of Web Samples:

The produced wet-laid non-woven fabrics were tested with regard to the parameters strength, width of the test specimen being 1.5 cm, and thickness. Strength index was calculated with the following formula: strength index=strength [cN]/basis weight [g/m²]. The results are shown in Table 10.

TABLE 10
Testing results of samples of example 5

| Sample | 5a | 5b | 5c | 5d |
|---|---|---|---|---|
| Tensile strength index | 15.0 | 29.7 | 18.0 | 27.2 |
| MD-dry [cN*m²/g] | | | | |
| Tensile strength index | 7.7 | 15.5 | 11.6 | 17.3 |
| CD-dry [cN*m²/g] | | | | |
| Tensile strength index | 4.6 | 9.0 | 6.2 | 9.4 |
| MD-wet [cN*m²/g] | | | | |
| Tensile strength index | 2.5 | 5.3 | 4.0 | 5.8 |
| CD-wet [cN*m²/g] | | | | |
| Basis weight [g/m²] | 40.4 | 40.5 | 41.0 | 41.3 |
| Air permeability [L/m²s] | 924 | 649 | 780 | 599 |
| Thickness [mm] | 0.308 | 0.285 | 0.305 | 0.296 |

Sample 5a) serves as reference sample for a wet-laid non-woven fabric with addition of viscose fibres suitable/good enough for further processing.

Samples 5b)-5d) show the benefit of increased dry and wet strength by addition of MFC. In the samples 5b and 5c, using MFC 1 and MFC 4 respectively, the strength increase is in the range of +100%. In sample 5c using the somewhat coarser MFC 3 the strength increase is only +35% (dry) and +50% (wet) underlining the importance to use MFC in an optimum particles size distribution.

1. A wetlaid web, selected from the group consisting of wet-laid non-woven fabrics and paper, comprising:
a cellulosic fibre material in the form of viscose fibre at an amount of at least 5% w/w, wherein the wetlaid web comprises microfibrillated cellulose at an amount of 0.5% w/w to 5% w/w, wherein the microfibrillated cellulose has a particle size distribution (x₁₀) of 5 μm to 30 μm, and
a wet-strength agent.

2. The wetlaid web according to claim 1, wherein the viscose fibre is selected from the group consisting of viscose fibres with a standard cross-section, viscose fibres with a flat cross-section, and mixtures thereof.

3. The wetlaid web according to claim 2, wherein at least a part of the viscose fibre is a solid viscose fibre with a flat cross-section having the following properties:
the ratio of width B to thickness D of the fibre is 10:1 or higher,
the fibre surface is essentially smooth, and
the fibre is essentially transparent.

4. The wetlaid web according to claim 1, wherein the amount of viscose fibre is 5% w/w to 95% w/w.

5. The wetlaid web according to claim 1, wherein the amount of viscose fibre is 50% w/w or more.

6. The wetlaid web according to claim 1, wherein the wetlaid web comprises, in addition to the viscose fibre, a further cellulosic fibre material.

7. The wetlaid web according to claim 6, wherein the fibre material comprised in the wetlaid web consists essentially of cellulosic fibre material.

8. The wetlaid web according to claim 1, wherein the length of the viscose fibre is from 0.1 mm to 16 mm.

9. The wetlaid web according to claim 1, wherein the microfibrillated cellulose has a particle size distribution (x₁₀) of 10 μm to 30 μm.

10. The wetlaid web according to claim 1, wherein the amount of wet-strength agent is 0.1% w/w to 5% w/w.

11. The wetlaid web according to claim 1, wherein the wet-strength agent is selected from the group consisting of polyamine epichlorhydrin resins (PAE) and polyamidamine epichlorhydrin resins (PAAE).

12. The wetlaid web according to claim 1, wherein the value of the wet-strength of the wetlaid web is at least 18% of the value of the dry-strength.

13. A fleece for food filtration comprising a wetlaid web according to claim 1.

14. A transparent cosmetic mask comprising a wetlaid web according to claim 1.

15. The wetlaid web according to claim 4, wherein the amount of viscose fibre is 5% w/w to 50% w/w, or 10% w/w to 30% w/w.

16. The wetlaid web according to claim 5, wherein the amount of viscose fibre is 80% w/w or more, or 95% w/w or more.

17. The wetlaid web according to claim 6, wherein the further cellulosic fibre comprises wood pulp.

18. The wetlaid web according to claim 8, wherein the length of the viscose fibre is from 3 mm to 12 mm, or from 4 mm to 8 mm.

19. The wetlaid web according to claim 9, wherein the microfibrillated cellulose has a particle size distribution (x₁₀) of 12 μm to 28 μm, or 12 μm to 25 μm.

20. The wetlaid web according to claim 10, wherein the amount of wet-strength agent is 0.5% w/w to 3% w/w, or 1% w/w to 3% w/w.

21. The wetlaid web according to claim 12, wherein the value of the wet-strength of the wetlaid web is at least 20% of the value of the dry-strength.
